(19) United States
(12) Patent Application Publication
(10) Pub. No.: US 2017/0252152 A1
(43) Pub. Date: Sep. 7, 2017

(54) SELF-CENTERING GUIDE CATHETER

(71) Applicants: BOSTON SCIENTIFIC SCIMED, INC., Maple Grove, MN (US); MAYO FOUNDATION FOR MEDICAL EDUCATION AND RESEARCH, Rochester, MN (US)

(72) Inventors: Gurpreet S. Sandhu, Rochester, MN (US); Daniel B. Spoon, Rochester, MN (US); Brandon J. Tefft, Rochester, MN (US); Roger W. McGowan, Ostego, MN (US); Patrick A. Haverkost, Corcoran, MN (US); Joel N. Groff, Delano, MN (US); LLoyd Radman, Blaine, MN (US); Daniel Ross, Watertown, MN (US)

(73) Assignees: BOSTON SCIENTIFIC SCIMED, INC., MAPLE GROVE, MN (US); MAYO FOUNDATION FOR MEDICAL EDUCATION AND RESEARCH, Rochester, MN (US)

(21) Appl. No.: 15/438,263

(22) Filed: Feb. 21, 2017

Related U.S. Application Data
(60) Provisional application No. 62/303,858, filed on Mar. 4, 2016.

(51) Int. Cl. A61F 2/24; A61M 25/09; A61M 25/01; A61B 17/12; A61M 25/00

(52) U.S. Cl. CPC A61F 2/243; A61F 2/2436; A61B 17/12122; A61B 17/12168; A61M 25/0074; A61M 25/0125; A61M 25/09; A61B 2017/22068

(57) ABSTRACT

An example medical device is disclosed. The device includes a tubular member having a lumen defined therein and a distal end portion, the distal end portion defining a guidewire port. The medical device also includes an expandable frame disposed along the distal end portion of the tubular member, the expandable frame being designed to shift between a first configuration and an expanded configuration. The frame includes a base, an end region and a plurality of struts extending between the base and the end region, the struts defining a plurality of apertures along the frame. The medical device also includes a cover attached to a portion of the frame and the cover is configured to cover one or more of the plurality of apertures such that the frame will align the guidewire port with an opening in a heart valve when the frame is positioned within a body lumen.

### RELATED APPLICATIONS

This application claims priority to U.S. Provisional Application Ser. No. 62/303,858, filed Mar. 4, 2016.

### TECHNICAL FIELD

The present disclosure pertains to medical devices, and methods for manufacturing medical devices. More particularly, the present disclosure pertains to elongated medical devices with self-centering capabilities.

### BACKGROUND

Cardiac valvular stenosis is a condition in which the heart's valves are narrowed (stenotic). For example, mitral valve stenosis is an abnormal narrowing of the mitral valve, resulting in a restriction of the blood flow from the left atrium to the left ventricle. Aortic valve stenosis occurs when the heart's aortic valve narrows, causing the heart to work harder to pump blood to the body. While medications can ease symptoms of mild to moderate aortic valve stenosis, the only way to treat severe aortic valve stenosis is via a procedure to replace the valve. Therapies to repair or replace the aortic valve include, among others, transcatheter aortic valve replacement (TAVR). TAVR involves replacing the aortic valve with a prosthetic valve that is delivered via the femoral artery, axillary or subclavian, for example.

Often, a diseased heart valve includes significant calcification surrounding the valve leaflets. The calcification may effectively alter the configuration of the native heart. For example, in a healthy heart, the valve opening is generally oriented in the center of the heart valve leaflets. However, calcification(s) may effectively “shift” the location of and/or narrow the valve opening. One of the most challenging steps when performing a TAVR, for example, is to locate the valve opening and pass a guidewire through the severely stenotic valve.

Current practice often entails a physician probing a guidewire across the surface of the valve in an attempt to locate the valve opening. By probing the surface of the valve, the clinician hopes to locate and cross the stenotic valve orifice. However, the high-pressure jet of blood passing through the valve, along with the movement of the heart, make the probing technique even more challenging. Further, the probing technique increases total surgery time, thereby prolonging a patient's exposure to radiation, imaging contrast agent, and anesthesia. Additionally, the probing technique increases the risk of dislodging calcified debris from the valve surface. Therefore, it may be desirable for a clinician to utilize a guidewire centering device which efficiently centers the guidewire over the valve opening prior to guidewire advancement.

The present disclosure relates to methods and apparatus for endovascular replacement of a heart valve. In particular, the present disclosure relates to methods and apparatus for centering a guidewire over the opening of a diseased heart valve.

### BRIEF SUMMARY

This disclosure provides design, material, manufacturing method, and use alternatives for medical devices. An example medical device include a tubular member having a lumen defined therein and a distal end portion, the distal end portion defining a guidewire port. The medical device also includes an expandable frame disposed along the distal end portion of the tubular member, the expandable frame being designed to shift between a first configuration and an expanded configuration. The frame includes a base, an end region and a plurality of struts extending between the base and the end region, the struts defining a plurality of apertures along the frame. The medical device also includes a cover attached to a portion of the frame and the cover is configured to cover one or more of the plurality of apertures such that the frame will align the guidewire port with an opening in a heart valve when the frame is positioned within a body lumen.

Alternatively or additionally to any of the embodiments above, wherein the end region is radially expanded when the frame is in the expanded configuration.

Alternatively or additionally to any of the embodiments above, wherein the frame has a conical shape when the frame is in the expanded configuration.

Alternatively or additionally to any of the embodiments above, wherein the frame includes a nickel-titanium alloy.

Alternatively or additionally to any of the embodiments above, wherein the cover is configured to funnel blood flow toward the plurality of apertures.

Alternatively or additionally to any of the embodiments above, wherein the cover extends circumferentially around the frame.

Alternatively or additionally to any of the embodiments above, wherein the cover is positioned adjacent to the end region of the frame.

Alternatively or additionally to any of the embodiments above, wherein a section of the frame adjacent to the base of the frame is free of the cover.

Alternatively or additionally to any of the embodiments above, wherein the cover includes a fabric.

Alternatively or additionally to any of the embodiments above, wherein the cover is disposed along an outer surface of the frame, and inner surface of the frame, or both.

Alternatively or additionally to any of the embodiments above, wherein the cover includes expanded polytetrafluoroethylene.

Alternatively or additionally to any of the embodiments above, further comprising an insertion sleeve disposed about the tubular member

Alternatively or additionally to any of the embodiments above, wherein the insertion sleeve is slidable relative to the tubular member, and wherein the insertion sleeve is capable of being slid over the frame in order to shift the frame to the first configuration.

Alternatively or additionally to any of the embodiments above, further comprising a marker disposed along a proximal region of the tubular member.

An example method for manufacturing a medical device includes:

attaching an expandable frame to a tubular member;

wherein the tubular member has a lumen defined therein and a distal end portion, the distal end portion defining a guidewire port;

wherein the expandable frame is designed to shift between a first configuration and an expanded configuration;

wherein the frame include a base, an end region and a plurality of struts extending between the base and the end region, the struts defining a plurality of apertures along the frame;

attaching a cover to a portion of the frame; and

wherein the cover is configured to cover one or more of the plurality of apertures such that the frame will align the guidewire port with an opening in a heart valve when the frame is positioned within a body lumen.

Another example self-centering medical device includes:

an elongate shaft having a guidewire lumen defined therein and having a distal guidewire port;

a sleeve slidably disposed about the shaft;

a self-expanding basket coupled to the shaft, the basket being designed to shift between a first configuration and an expanded configuration;

wherein the sleeve is disposed about the basket when the basket is in the first configuration;

wherein the basket include a base positioned proximal of the distal guidewire port, an end region positioned adjacent to the distal guidewire port, and a plurality of struts extending between the base and the end region, wherein the struts define a plurality of apertures along the frame;

a cover attached to the frame; and

wherein the cover is configured to cover some of the plurality of apertures such that when the frame is positioned in a blood vessel adjacent to a heart valve, the flow of blood will be directed toward the plurality of apertures such that the distal guidewire port with align with an opening in the heart valve.

Alternatively or additionally to any of the embodiments above, wherein the basket has a conical shape when in the expanded configuration.

Alternatively or additionally to any of the embodiments above, wherein the cover extends circumferentially about a distal portion of the basket and wherein a proximal portion of the basket is free of the cover.

Alternatively or additionally to any of the embodiments above, wherein the cover is disposed along an outer surface of the basket, and inner surface of the basket, or both.

Alternatively or additionally to any of the embodiments above, wherein the cover includes expanded polytetrafluoroethylene.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example intravascular medical device in an expanded configuration;

FIG. 2 is a side view of an example intravascular medical device in a collapsed configuration;

FIG. 3 is an example illustration of positioning an example intravascular medical device in a patient;

FIG. 4 is an example illustration of an example intravascular device positioned in the heart;

FIG. 5 is a perspective view of another example intravascular medical device in an expanded configuration;

FIG. 6 is an end view of another example intravascular medical device in an expanded configuration;

FIGS. 7 and 8 illustrate an example intravascular medical device positioned in a body lumen.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

As discussed above, a diseased heart valve includes significant calcification surrounding the valve leaflets. The calcification may effectively alter the configuration of the native heart. For example, in a healthy heart, the valve opening is generally oriented in the center of the heart valve leaflets. However, calcification(s) may effectively “shift” the location of and/or narrow the valve opening. One of the most challenging steps when performing a TAVR, for example, is to locate the valve opening and pass a guidewire through the severely stenotic valve.

Current practice often entails a physician probing a guidewire across the surface of the valve in an attempt to locate the valve opening. By probing the surface of the valve, the clinician hopes to locate and cross the stenotic valve orifice. However, the high-pressure jet of blood passing through the valve, along with the movement of the heart, make the probing technique even more challenging. Further, the probing technique increases total surgery time, thereby prolonging a patient's exposure to radiation, imaging contrast agent, and anesthesia. Additionally, the probing technique increases the risk of dislodging calcified debris from the valve surface. Therefore, it may be desirable for a clinician to utilize a guidewire centering device which efficiently centers the guidewire over the valve opening prior to guidewire advancement. The following disclosure relates to methods and apparatus for centering a guidewire over the opening of a diseased heart valve. In particular, that following examples disclose an intravascular medical device including a covered basket attached to a flexible shaft, whereby the covered basket is designed to self-center on blood flowing through a diseased heart valve.

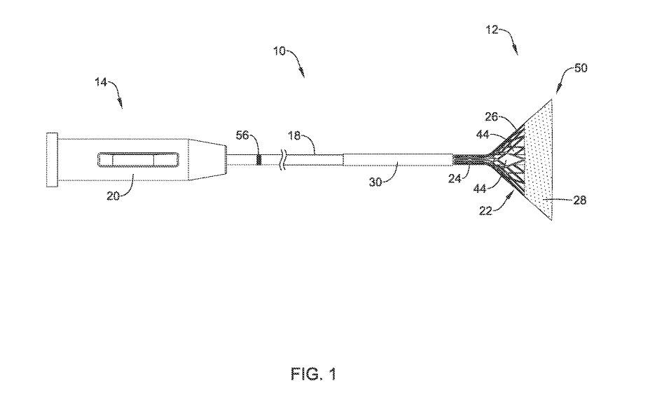

FIG. 1 illustrates an example intravascular medical device 10. Intravascular medical device 10 may be configured to be positioned in a body lumen for a variety of medical applications. For example, intravascular medical device 10 may be used to position a device such as an artificial valve or filter within a body lumen, in some instances.

An example first step to utilize medical device 10 to replace a heart valve may include gaining access to the body via the vasculature. For example, a clinician may initially place a guidewire in the aortic arch via the femoral artery, axillary or subclavian, for example. Next, a clinician may use the guidewire to place a guide catheter up and over the aortic arch. The clinician may place the guide catheter substantially adjacent an outflow portion of the aortic valve, for example. The clinician may then advance intravascular medical device 10 through the lumen and out the distal end of the guide catheter. Using medical device 10 to center the guidewire over the valve opening, the clinician may then advance the guidewire through the valve opening and into the left ventricle. While maintaining the position of the guidewire in the left ventricle, the clinician may then recapture medical device 10 within the guide catheter. In some instances, the clinician may advance the guide catheter (including recaptured medical device 10) into the left ventricle. Once positioned within (e.g., prolapsed within) the left ventricle, the clinician may exchange the original guidewire for a second guidewire. The clinician may then retract the medical device 10 and guide catheter (while maintaining the position of the second guidewire) out of the patient and use the second guidewire to advance additional medical devices into the heart.

The medical device 10 of FIG. 1 may include a shaft 18 having a proximal end portion 14 and a distal end portion 12. Shaft 18 may be defined as a tubular member including lumen extending therein. In other words, shaft 18 may include a lumen which extends along the entire length of shaft 18 or the lumen may extend along only a portion of shaft 18. The lumen of shaft 18 may be sized and/or shaped to accommodate a guidewire to extend therein.

The proximal portion 14 of shaft 18 may include a hub member 20 attached thereto. Hub member 20 may include a lumen and/or passage extending therethrough which substantially aligns with the lumen of shaft 18. Similar to that described with respect to shaft 18, it can be appreciated that the lumen of hub 20 may be sized and/or shaped to accommodate a guidewire extending therethrough. The shape of hub 20 illustrated in FIG. 1 is just one of a variety of shapes and/or configurations that can perform the functions disclosed herein.

Shaft 18 may include a distal end region 12. A basket member 50 may be disposed along the distal end region 12 of shaft 18. Basket member 50 may generally include frame 22. In some instances, frame 22 may be formed (e.g., laser cut) from a metallic (e.g., Nitinol) tube. For example, in some instances basket 50 may be defined as a self-expanding shape memory basket. However, it is contemplated that frame 22 may be constructed from materials other than Nitinol. For example, frame 22 may be constructed from alternative metals, polymeric and/or ceramic materials and combinations thereof.

In some examples, frame 22 may include one or more strut members 26 arranged to form a scaffold. As will be described in greater detail below, strut members 26 may be arranged to form a scaffold having a variety of the patterns, shapes, geometries, etc. Additionally, it can be appreciated that strut members 26 may generally be arranged such that frame 22 may be defined as extending completely around (e.g., 360 degrees) the outer surface of shaft 18.

Basket member 50 (including frame member 22) may further define a base member 24. Base member 24 may be a portion (e.g., unitary member) of the tube (e.g., Nitinol tube) used to form frame 22 described above. Alternatively, it is contemplated that base member 24 may be a distinct component attached separately to frame 22. For example, base member 24 may be welded, joined, etc. to one or more strut members 26 defining frame 22. Additionally, base member 24 may be attached to shaft 18. In some examples, strut members 26 may form a unitary structure with base member 24. Further, strut members 26 may be positioned between base member 24 and frame 22.

In some examples, basket member 50 may further include a covering 28 (depicted by the dotted pattern in FIG. 1) disposed along and/or attached to the entire frame 22 or along only a portion of frame 22. In other words, covering 28 may cover the entire surface area of the frame 22 or may cover only a portion of frame 22. As will be described in greater detail below, covering 28 may cover only selected portions of frame 22 thereby creating portions of frame 22 which are free from covering 28. The portions of frame 22 which are free of covering 28 may be characterized as apertures, openings, holes, etc. 44 in basket member 50. However, it is contemplated that the entire surface of the frame 22 may include a covering 28 in conjunction with selected portions of the covering 28 being removed to form apertures, openings, holes, etc. 44 in basket member 50. For example, any surface of any portion of strut members 26 may include a covering 28 while the apertures 44 formed between strut members 26 may be free of the covering 28.

Covering 28 may be formed from a suitable material. For example, covering 28 may include silicone, polytetrafluoroethylene, polyurethane, or the like, or other materials including those disclosed herein. In some instances, cover member 28 may be disposed along an outer surface of frame 22. In other instances, cover member 28 may be disposed along both an inner and an outer surface of frame 22. In some of these and in other instances, cover member 28 may encapsulate (e.g., laminate) frame 22 or otherwise have frame 22 embedded therein. Coupling cover 28 to frame 22 may include laminating, thermal bonding, molding, coating, dip coating, extruding, or the like. Additionally, while not shown in FIG. 1, any portion of covering 28 may include a slit, cut, hole, aperture, etc. which may permit fluid to flow therethrough. For example, a portion of covering 28 may have a “T-shaped” slit that opens to permit blood to flow therethrough. This is just an example and not intended to be limiting as other aperture designs are contemplated.

FIG. 1 further illustrates that medical device 10 may include one or more markers 56. Markers 56 may or may not include a radiopaque material. For example, FIG. 1 shows marker 56 disposed (e.g., attached, fixed, positioned) along shaft member 18. However, while FIG. 1 shows marker 56 disposed along shaft 18, it is contemplated that markers 56 may be disposed on other portions of medical device 10. For example, markers may be disposed on basket member 50 and/or hub 20. Marker 56 may be used to visualize the relative position of medical device 10 with respect to a patient's heart and/or additional medical devices used in a medical procedure. For example, marker 56 may be used to identify when basket 50 is at the distal end of a guide catheter and about to be deployed therefrom.

Additionally, FIG. 1 shows insertion sleeve 30 disposed along shaft 18. Insertion sleeve 30 may be defined generally as a tubular member having length and a lumen extending therethrough. In some examples, the dimensions (e.g., diameter of the lumen) of insertion sleeve 30 may be designed such that shaft 18 may pass through the lumen of insertion sleeve 30. Further, it can be appreciated that insertion sleeve 30 may be designed such that it can slide and/or translate along shaft 18.

Sleeve 30 may be designed such that it may slide toward the distal end 12 of shaft 18. Additionally, in some examples insertion sleeve may be designed such that it can slide over basket member 50. For example, basket 50 may be designed such that it can radially collapse from the expanded configuration shown in FIG. 1 to a collapsed configuration. In other words, strut members 26 of frame 22 may fold radially inward toward a longitudinal axis of shaft 18. It can be appreciated the expanded portion of frame 22 may collapse to an outer diameter that is substantially equivalent to (e.g., be flush with) the outer diameter of base member 24.

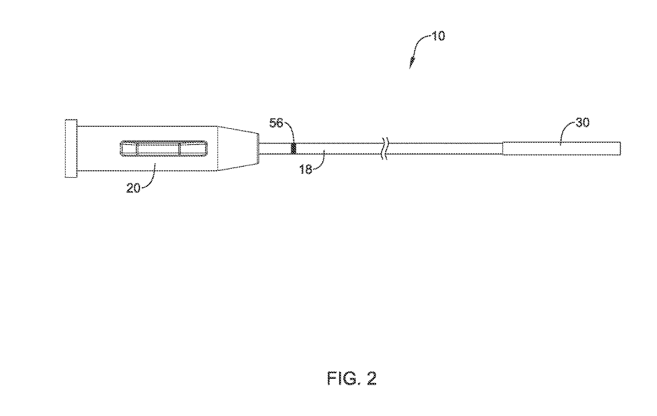

As stated above, in some instances it may be desirable to shift basket member 50 from an expanded configuration to a collapsed configuration. For example, it may be desirable to position basket member 50 in a collapsed configuration to assist a clinician in advancing and/or positioning medical device 10 in a patient. FIG. 2 illustrates medical device 10 in a collapsed configuration. As shown in FIG. 2, insertion sleeve 30 has been advanced (e.g., slid, translated, etc.) toward the distal portion of shaft 18. Further, insertion sleeve 30 has been positioned (e.g., slid) over basket member 50. While not shown in FIG. 2, it can be appreciated that basket member 50 is collapsed and restrained under insertion sleeve 30. Accordingly, it can further be appreciated that sliding insertion sleeve toward the proximal region of shaft 18 may permit basket member 50 to radially expand. In other words, translating insertion sleeve along shaft 18 toward the proximal end of shaft 18 may uncover basket member 50.

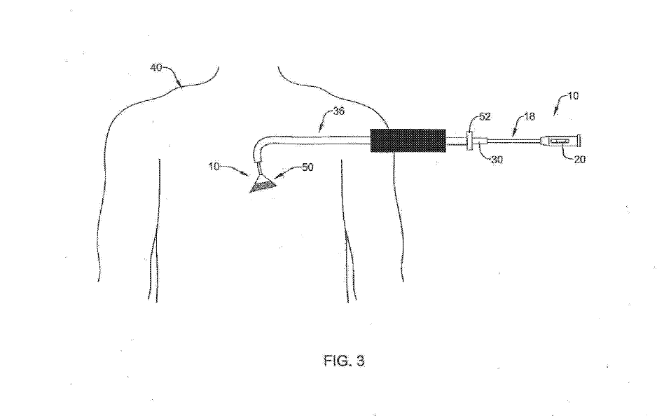

FIG. 3 depicts an example delivery system and methodology for medical device 10. FIG. 3 shows an example guide catheter 36 extending from a position outside a patient 40 to a position inside a patient 40. It can be appreciated that example guide catheter 36 may include a distal end (positioned inside a patient), a proximal end (nearest a clinician) and a lumen extending within the guide catheter from the proximal to distal end. Further, guide catheter 36 may include a hub member 52 disposed along the proximal end of the guide catheter 36. Hub 52 may include a luer or other type of connector. Hub 52 may also include a hemostatic valve (not shown). FIG. 3 further illustrates that medical device 10 may be inserted into and extend through the lumen of guide catheter 36. As depicted in FIG. 3, medical device 10 may be advanced out of the distal end of the guide catheter 36 while positioned inside a patient.

It can be appreciated that in some instances it may be difficult to insert basket member 50 into the hub 52 of guide catheter 36 while basket 50 is in an expanded configuration. Therefore, in order to allow a clinician to easily insert medical device 10 into guide catheter 36, it may be desirable to insert basket member 50 into the hub 52 of guide catheter 36 while basket member 50 is in a collapsed configuration.

An example methodology of inserting medical device 10 into guide catheter 36 may include using insertion sleeve 30 to effectively “bridge” hub 52 of guide catheter 36. In other words, while basket member 50 is held in a collapsed configuration via insertion sleeve 30, insertion sleeve 30 is partially inserted into hub 52 of guide catheter 36. It can be appreciated that “partially inserting” insertion member 30 into guide catheter 36 may include abutting hub 52 with insertion member 30, partially inserting insertion member 30 into hub 52, or partially inserting insertion member 30 through hub 52 such that a portion of insertion member 30 is positioned within the lumen of guide catheter 36. However, despite the degree to which insertion member 30 is advanced into and/or through hub 52, a portion of insertion sleeve 30 remains proximal (e.g., outside) hub 52.

Once insertion sleeve 30 is sufficiently positioned within guide catheter 36, a clinician may grasp the portion of the insertion sleeve 30 extending outside hub 52. While holding insertion sleeve stationary relative to guide catheter 36, a clinician may advance medical device 10 distally. It can be appreciated that this distal advancement will effectively push medical device 10 out of the distal end of the insertion sleeve 30. However, because the distal end of insertion sleeve 30 is positioned within the hub 52 and/or lumen of guide catheter 36, medical device 10 will also be positioned within the hub 52 and/or lumen of guide catheter 36. The clinician may then continue to advance medical device 10 in a distal direction until basket member 50 exits the distal end of guide catheter 36 adjacent a target site.

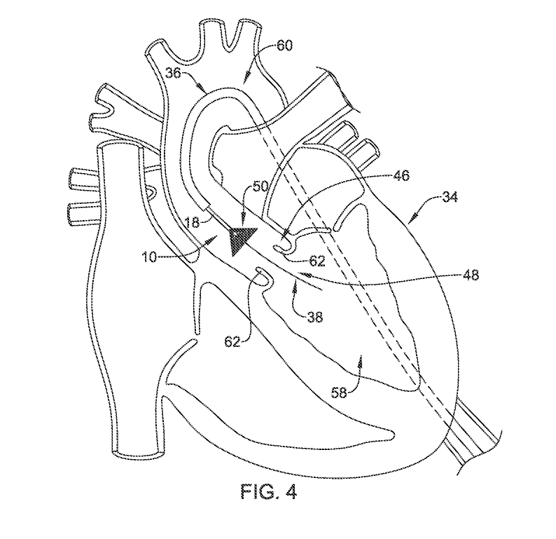

FIG. 4 shows example guide catheter positioned in the aorta 60 of a patient. Additionally, FIG. 4 shows medical device 10 positioned adjacent heart valve 46 after having been advanced through guide catheter 36 and exiting (e.g., advancing, deploying, etc.) out of the distal end of the guide catheter 36. As shown, heart 34 may include a heart valve 46 having valve leaflets 62. Heart valve leaflets 62 may define a valve opening 48 (e.g., an opening defined by valve leaflets 62). It can be appreciated from FIG. 4 that heart valve leaflets are opened toward basket member 50. In other words, as blood is pumped from the left ventricle 58 into the aorta 60, the blood flows from the left ventricle 58, though leaflets 62 and toward basket member 50 of medical device 10. FIG. 4 further illustrates a guidewire 38 extending from the lumen of shaft 18 of medical device 10. Guidewire 38 may be advanced away from medical device 10, through heart valve 46 (e.g., through valve opening 48 and into left ventricle 58) and into the left ventricle 58.

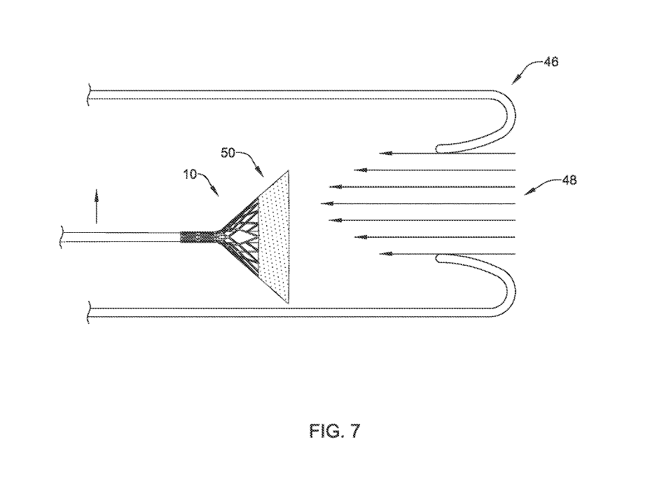

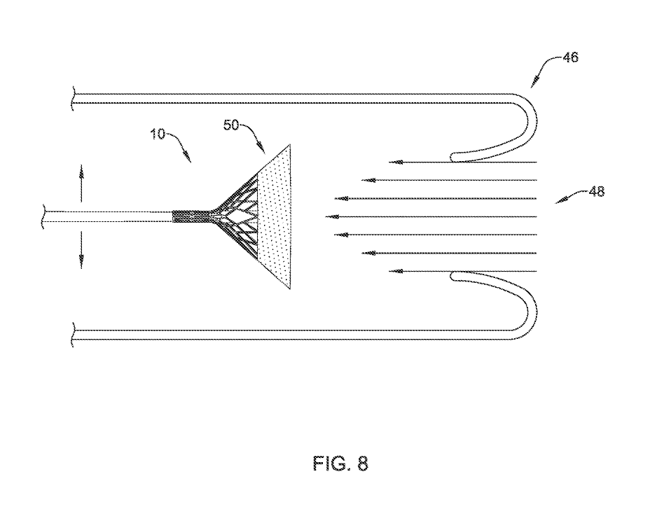

As stated above, basket member 50 of medical device 10 is designed to self-align over an opening of a diseased heart valve. Specifically, medical device 10 is designed such that the shape of basket 50, in combination with a covering 28 and apertures 44, is drawn to the flow of blood passing through a valve opening. In particular, basket 50 is designed such that it self-centers on the fastest portion of a bloodstream. As will be discussed later with respect to FIGS. 7-8, if a bloodstream were represented via a series of vectors representing its velocity, basket member 50 would self-align along the vector with the greatest magnitude.

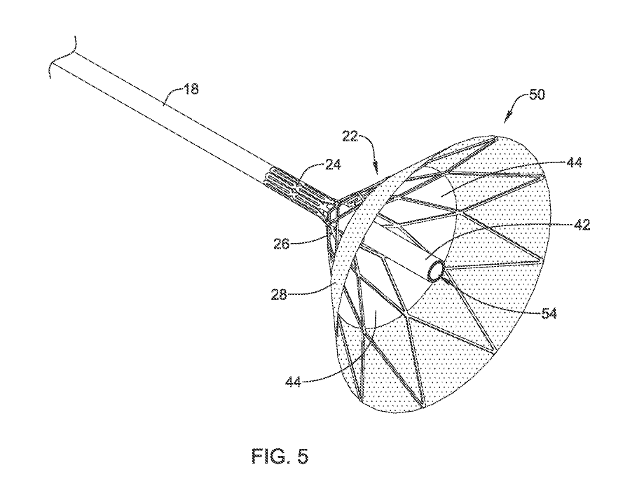

As alluded to above, the ability of basket 50 to self-center within a bloodstream may correspond to the geometric distribution of strut members 26 in combination with covering 28 and apertures 44. FIG. 5 is a perspective view of an example basket member 50 including struts 26, apertures 44 and covering 28. It can be appreciated that the basket member 50 shown in FIG. 5 is substantially conical shaped. It can be further appreciated that the general conical shape of basket 50 may facilitate the basket's 50 ability to be “pulled into” a flow of blood. In other words, the conical shape of basket 50 may increase its ability to “catch” a fast-moving bloodstream at its edge and “funnel” all or a portion of that bloodstream from its edge radially inward toward apertures 44. Further, it can be appreciated that a bloodstream may continue to enter the basket 50, be funneled toward apertures 44 and exit through apertures 44. In essence, the fastest portion of a bloodstream may hold a basket 50 in place, and therefore, maintain medical device 10 centered on a valve opening as the bloodstream continues to flow through valve.

While FIG. 5 shows a frame 22 having apertures shaped generally as diamonds, it can be appreciated that frame 22 may include a variety of shape, sizes, designs, arrangements, geometries, etc. Further, in some examples, frame 22 may include a variety of shapes and/or geometric arrangements. For example, while the above discussion has focused on the shape of frame 22 shown in FIG. 5, it is not intended to be limiting. For example, strut members 26 may be arranged within frame 22 (e.g., within basket 50) such that they create one or more apertures 44. The number, shape, configuration and/or arrangement of strut members 26 and/or apertures 44 may depend on the particular performance characteristics desired to be balanced within basket 50. For example, additional strut members 26 may be added to frame 22 to provide increased stiffness to basket 50. In other instances, strut members 26 may take on particular geometries that increase or decrease (e.g., adjust) how blood flows through basket 50, for example.

Similarly, covering 28 may be located (e.g., arranged) throughout frame 22 in a variety of configurations to provide a tailored response to a bloodstream and/or blood vessel. In other words, a wide variety of different shapes and/or arrangements of struts 22 and covering 28 may be included within basket 50 in order to impart customized performance characteristics to medical device 10. For example, in some instances it may be desirable to include covering 28 over a greater percentage of apertures 44. However, in other instances it may be desirable to cover a lower percentage of apertures 44. As discussed, numerous different frame 22, aperture 44 and covering combinations may be contemplated to impart a specific performance onto medical device 10. For example, in some instances covering 28 may be disposed substantially along the distal portion 12 (e.g., along a distal end region) of frame 22 (as shown in FIG. 1, for example).

FIG. 5 further shows guidewire port 42 positioned substantially in the center of basket 50. Guidewire port 42 may be defined as the distal end of shaft 18. Further, FIG. 5 shows lumen 54 of shaft 18. Lumen 54 may be designed such that a guidewire may be advanced therethrough. In some examples, the distal end of guidewire port 42 may not extend beyond the distal end of basket 50. In other examples, the distal end of guidewire port 42 may be substantially flush with the distal end of basket 50. In yet other examples, the distal end of guidewire port 42 may extend distally beyond the end of basket 50.

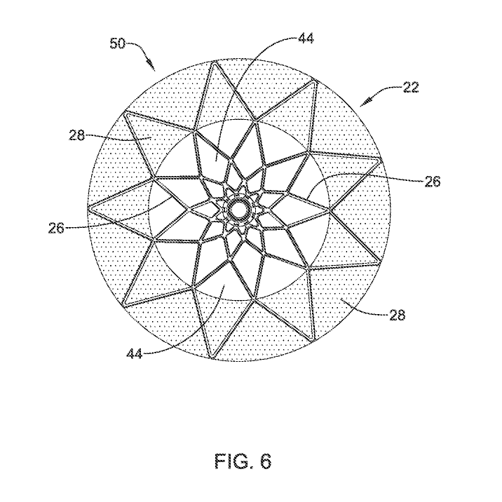

FIG. 6 is an end view of an example basket 50 having an example frame geometry 22 and covering 28. As shown in FIG. 6, struts 26 are arranged such that they create generally diamond-shaped apertures 44. Further, it can be seen that apertures 44 decrease in size when moving from the outer edge (e.g., outer circumference) to the center of basket 50. These are only examples. As stated above, numerous geometries, patterns and covering design combinations are contemplated.

FIGS. 7 and 8 provide illustrations of the above discussion regarding how medical device 10 self-centers within a bloodstream. FIG. 7 shows an example valve 46 including a valve opening 48. Further, as blood flows through valve 46, a gradient of velocity vectors having variable magnitudes may be observed corresponding to the flow of the bloodstream. As shown in FIG. 7, an example medical device 10 may initially be deployed offset from the velocity vector having the greatest magnitude (corresponding to that portion of the bloodstream having the greatest velocity). However, as can be seen in FIG. 7, a portion of the outer edge of basket 50 may “capture” the largest velocity vector. That portion of the bloodstream may then effectively “pull” basket 50 toward the center of valve opening 48 (as depicted by the arrow in FIG. 7), thereby, centering basket 50 over the valve opening.

Once centered over the valve opening (and, correspondingly, the velocity flow vector having the greatest magnitude), basket 50 will remain centered over that portion of the bloodstream having the greatest velocity. It can be appreciated that the fastest moving portion of the bloodstream may correlate with the valve opening. Therefore, the basket 50 may change its position in response to a change in position of the velocity vectors, as depicted by the upward and downward pointing arrows shown in FIG. 8. In other words, the center of basket 50 will generally self-align with the fastest moving portion of a bloodstream flowing through a valve opening. Because the guidewire port is located substantially in the center of basket 50, a guidewire being advancing through the guidewire port will be advanced directly through the valve opening.

The materials that can be used for the various components of device 10 (and/or other devices disclosed herein) and the various tubular members disclosed herein may include those commonly associated with medical devices. For simplicity purposes, the following discussion makes reference to device 10 and other components of device 10. However, this is not intended to limit the devices and methods described herein, as the discussion may be applied to other similar tubular members and/or components of tubular members or devices disclosed herein.

Device 10 and/or other components of device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of device 10 may also be doped with, made of, or otherwise include a radiopaque material. Radiopaque materials are understood to be materials capable of producing a relatively dark image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 10 to achieve the same result.

In some embodiments, a degree of Magnetic Resonance Imaging (MRI) compatibility is imparted into device 10. For example, device 10, or portions thereof, may be made of a material that does not substantially distort the image and create substantial artifacts (e.g., gaps in the image). Certain ferromagnetic materials, for example, may not be suitable because they may create artifacts in an MRI image. device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device, comprising:
a tubular member having a lumen defined therein and a distal end portion, the distal end portion defining a guidewire port;
an expandable frame disposed along the distal end portion of the tubular member, the expandable frame being designed to shift between a first configuration and an expanded configuration;
wherein the frame include a base, an end region and a plurality of struts extending between the base and the end region, the struts defining a plurality of apertures along the frame;
a cover attached to a portion of the frame; and
wherein the cover is configured to cover one or more of the plurality of apertures such that the frame will align the guidewire port with an opening in a heart valve when the frame is positioned within a body lumen.

2. The medical device of claim 1, wherein the end region is radially expanded when the frame is in the expanded configuration.

3. The medical device of claim 1, wherein the frame has a conical shape when the frame is in the expanded configuration.

4. The medical device of claim 1, wherein the frame includes a nickel-titanium alloy.

5. The medical device of claim 1, wherein the cover is configured to funnel blood flow toward the plurality of apertures.

6. The medical device of claim 1, wherein the cover extends circumferentially around the frame.

7. The medical device of claim 1, wherein the cover is positioned adjacent to the end region of the frame.

8. The medical device of claim 1, wherein a section of the frame adjacent to the base of the frame is free of the cover.

9. The medical device of claim 1, wherein the cover includes a fabric.

10. The medical device of claim 1, wherein the cover is disposed along an outer surface of the frame, and inner surface of the frame, or both.

11. The medical device of claim 1, wherein the cover includes expanded polytetrafluoroethylene.

12. The medical device of claim 1, further comprising an insertion sleeve disposed about the tubular member.

13. The medical device of claim 12, wherein the insertion sleeve is slidable relative to the tubular member, and wherein the insertion sleeve is capable of being slid over the frame in order to shift the frame to the first configuration.

14. The medical device of claim 1, further comprising a marker disposed along a proximal region of the tubular member.

15. A method for manufacturing a medical device, the method comprising:
attaching an expandable frame to a tubular member;
wherein the tubular member has a lumen defined therein and a distal end portion, the distal end portion defining a guidewire port;
wherein the expandable frame is designed to shift between a first configuration and an expanded configuration;
wherein the frame include a base, an end region and a plurality of struts extending between the base and the end region, the struts defining a plurality of apertures along the frame;
attaching a cover to a portion of the frame; and
wherein the cover is configured to cover one or more of the plurality of apertures such that the frame will align the guidewire port with an opening in a heart valve when the frame is positioned within a body lumen.

16. A self-centering medical device, comprising:
an elongate shaft having a guidewire lumen defined therein and having a distal guidewire port;
a sleeve slidably disposed about the shaft;
a self-expanding basket coupled to the shaft, the basket being designed to shift between a first configuration and an expanded configuration;
wherein the sleeve is disposed about the basket when the basket is in the first configuration;
wherein the basket include a base positioned proximal of the distal guidewire port, an end region positioned adjacent to the distal guidewire port, and a plurality of struts extending between the base and the end region, wherein the struts define a plurality of apertures along the frame;
a cover attached to the frame; and
wherein the cover is configured to cover some of the plurality of apertures such that when the frame is positioned in a blood vessel adjacent to a heart valve, the flow of blood will be directed toward the plurality of apertures such that the distal guidewire port with align with an opening in the heart valve.

17. The self-centering medical device of claim 16, wherein the basket has a conical shape when in the expanded configuration.

18. The self-centering medical device of claim 16, wherein the cover extends circumferentially about a distal portion of the basket and wherein a proximal portion of the basket is free of the cover.

19. The self-centering medical device of claim 16, wherein the cover is disposed along an outer surface of the basket, and inner surface of the basket, or both.

20. The self-centering medical device of claim 16, wherein the cover includes expanded polytetrafluoroethylene.
